(19) United States
(12) Patent Application Publication
(10) Pub. No.: US 2023/0018712 A1
(43) Pub. Date: Jan. 19, 2023

(54) HYDROXYL ION GENERATOR APPARATUSES FOR CEILING MOUNT OR WALK THROUGH

(71) Applicant: TURNTEK, Indianapolis, IN (US)

(72) Inventors: Robert Edward Turner, Indianapolis, IN (US); Brian Whitney Williams, Indianapolis, IN (US)

(21) Appl. No.: 17/757,587

(22) PCT Filed: Sep. 20, 2021
(86) PCT No.: PCT/US2021/051069

§ 371 Date: Jun. 17, 2022

Related U.S. Application Data
(60) Provisional application No. 63/080,937, filed on Sep. 21, 2020.
(60) Provisional application No. 63/123,556, filed on Dec. 10, 2020.

(51) Int. Cl. A61L 2/20; A61L 2/26; A61L 2/00

(52) U.S. Cl. CPC A61L 2/20; A61L 2/26; A61L 2/0094; A61L 2202/11; A61L 2202/15

(57) ABSTRACT

The invention describes a method and a walk through apparatus to treat surfaces with hydroxyl ions to reduce the viability and/or kill pathogens.

### CROSS REFERENCE TO RELATED APPLICATIONS

This application claims the benefit of U.S. Provisional Pat. Application No. 63/123556, filed Dec. 10, 2020 and U.S. Provisional Pat. Application No. 63/080937, filed Sep. 21, 2020, and PCT Application No. PCT/US2021/51069, filed Sep. 20, 2021 the entire contents of which are incorporated herein by reference.

### FIELD OF THE INVENTION

The invention relates generally to an apparatus of sufficient dimensions such that an individual can pass through the apparatus wherein the apparatus generates sufficient hydroxyl ion content to treat and/or kill pathogens, such as protozoa, fungi, molds, viruses, bacteria, etc. on the individual or surfaces of objects passed through the apparatus. In another aspect, the invention relates generally to an apparatus that can be used in enclosed areas, such as office spaces, restaurants, bars, storage rooms, warehouses, classrooms, etc., by filtering air, passing the filtered air through a hydroxyl ion generator and distributing the hydroxyl ions into the enclosed area to reduce or eliminate pathogens, such as protozoa, fungi, molds, viruses, bacteria, etc. in the enclosed space and/or on the surfaces within the enclosed space.

### BACKGROUND OF THE INVENTION

Coronaviruses are a group of related RNA viruses that affect mammals and birds. In humans and birds, such viruses can cause respiratory tract infections that range from mild to lethal. Mild illnesses in humans include some cases of the common cold (which is also caused by other viruses, predominantly rhinoviruses), while more lethal varieties can cause SARS, MERS, and COVID-19 as well as COVID-19 variants. A few vaccines are currently being marketed to treat COVID-19, however, what is termed the “delta” variant appears to be more virulent and is causing some breakthrough cases where the vaccine is not completely effective.

In particular, COVID-19 and variants thereof, such as the delta and mu variants, also referred to as SARS-CoV-2 and the variants thereof, is thought to spread mainly from person to person, mainly through respiratory droplets produced when an infected person coughs or sneezes. These droplets can land in the mouths or noses of people who are nearby or possibly be inhaled into the lungs. It is thought that spread is more likely when people are in close contact with one another (within about 6 feet). It may be possible that a person can get COVID-19 or a variant by touching a surface or object that has the virus on it and then touching their own mouth, nose, or possibly their eyes, however, there is no definitive answer at this time.

An important issue associated with coronaviruses, such as COVID-19 and variants, is how to control the spread of the virus. This aspect is especially important to consider when individuals travel about the world via public transportation, airplanes, trains and the like. Additionally, questions exist as to how to control the spread of the virus in settings where there are often large gatherings of individuals such as schools, restaurants, office buildings, hospitals, clinics, emergency rooms and the like.

Therefore, a need exists for an efficient method and apparatus that can minimize and or eliminate airborne or surface deposited viruses.

### BRIEF SUMMARY OF THE INVENTION

The present embodiments surprisingly provides a method and apparatus to subject an object, such as an individual, to an atmosphere containing hydroxyl ions to reduce or eliminate unwanted pathogens, such as protozoa, fungi, molds, bacteria and/or viruses, such as corona viruses.

In one aspect, the embodiments described herein provide a portal comprising a plurality of orifices, e.g., nozzles, arranged in an array and positioned on at least one of a first and/or a second opposing side of the portal and a hydroxyl ion generator in communication with the portal and the plurality of orifices, such as nozzles. A vapor of hydroxyl ions is transmitted through the orifices to emit the vapor of hydroxyl ions into the area formed by the portal. An object, such as a person, can pass through the portal and be treated with hydroxyl ions to reduce or eliminate unwanted pathogens, such as protozoa, fungi, molds, bacteria and/or viruses. The apparatus and method provide a safe and effective approach to reducing and/or eliminating harmful pathogens. Hydroxyl ions are safe for use with humans/mammals and will not harm an individual or surface(s). This approach as the advantages of being very efficient in the destruction of unwanted pathogens, such as protozoa, fungi, molds, bacteria and/or viruses, short contact times for effectiveness, the ability to treat a surface without harm to the surface and, for example, does not harm plant life/vegetation.

In another aspect, the embodiments relate generally to an apparatus that can be used in enclosed areas, such as office spaces, restaurants, bars, storage rooms, warehouses, classrooms, etc. by filtering air, passing the filtered air through a hydroxyl ion and distributing the hydroxyl ions into the enclosed area to reduce the concentration and/or kill pathogens, such as protozoa, fungi, molds, viruses, bacteria, etc. in the enclosed space and/or on the surfaces within the enclosed space.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 depicts a cut away view of a portal apparatus system 10.

FIG. 2 depicts an exterior view of hollow side panel 20 of portal apparatus system 10 showing air filter 120.

FIG. 3 depicts an interior view of hollow side panel 20 of portal apparatus system 10 with orifices 130 shown.

FIG. 4 depicts an optional top portion 140 of portal apparatus system 10 with orifices 130 (e.g., nozzles).

FIG. 5 depicts a side view of portal apparatus system 10 showing side panels 20, the inflection point for radius 35 to optional top portion 140 and orifices 130.

FIG. 6 depicts a top and/or bottom view of an apparatus suitable for use in an enclosed area via a ceiling mount or as a stand-alone unit.

FIG. 7 depicts the enclosed area unit apparatus hydroxyl generating ion components/assembly.

### DETAILED DESCRIPTION

In the specification and in the claims, the terms “including” and “comprising” are open-ended terms and should be interpreted to mean “including, but not limited to.... ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Currently, the world is dealing with a pandemic due to the spread of COVID-19 and variants thereof. At present, there isn’t a drug regime to combat the effects of the virus let alone a particular vaccine that is 100% effective to prevent infection of the virus from person to person. Methods to treat objects that may have been exposed to viruses are somewhat limited in that efficacy of treatment is not fully understood. Constant cleaning of surfaces, face masks, social distancing, avoidance of large groups of people, etc. can only prevent transmission of viruses to a certain extent. Because of this uncertainty, mass transit travel has become limited, visits to hospitals, clinics, and/or emergency rooms have become questionable as to whether a healthy person may be subjected to unwanted viruses, etc. The result has been, and continues to be, a major disruption to businesses, the economy, health and safety considerations, health care, education, etc.

The present embodiments provide a safe, efficient, and rapid approach to decontaminate surfaces and/or the air so that objects can be transferred from site to site without concern whether the surface is contaminated with pathogens, such as protozoa, fungi, molds, bacteria and/or viruses. In one aspect, the object is an individual. Individuals can pass through a portal that provides a cloud/vapor of hydroxyl ions that will degrade and destroy pathogens, such as protozoa, fungi, molds, bacteria and/or viruses from the surface an object or from the individual. The process is safe and effective in the treatment of bacteria and viruses, such as COVID-19.

It should be understood that throughout this specification, reference to COVID-19 refers to infectious disease caused by the novel coronavirus, SARS-CoV-2, that appeared in late 2019 and also includes variants thereof. COVID-19 is predominantly a respiratory illness that can affect other organs. People with COVID-19 have reported a wide range of symptoms, ranging from mild symptoms to severe illness. Symptoms may appear 2 to 14 days after exposure to the virus. Symptoms may include: fever or chills; cough; shortness of breath; fatigue; muscle and body aches; headache; new loss of taste or smell; sore throat; congestion or runny nose; nausea or vomiting; diarrhea.

COVID-19 variants include, but are not limited to, the Alpha (B.1.1.7), Beta (B.1.351, B.1.351.2, B.1.351.3), Delta (B.1.617.2, AY.1, AY.2, AY.3), Gamma (P.1, P.1.1, P.1.2) and Mu (B.1.621).

In one aspect, the present embodiments provide an apparatus comprising:
a portal comprising a plurality of orifices arranged in an array and positioned on at least one of a first and/or a second opposing side of the portal; and
a hydroxyl ion generator in communication with the portal and the plurality of orifices, wherein a vapor of hydroxyl ions can be transmitted through the orifices to emit the vapor of hydroxyl ions into the area formed by the portal.

The portal can further include a top portion that is connected to the first and second opposing sides. The top portion can further include a plurality of orifices, wherein the vapor of hydroxyl ions can be transmitted through the top portion orifices in addition to the first and second side opposing side orifices.

The term “portal” is meant to be an open enclosure that has at least two sides, a first and second opposing side. The sides provide a conduit for the transfer of hydroxyl ions through the enclosure. That is, the hollow sides function as ductwork, a conduit or a tube to deliver the hydroxyl ions to and through the plurality of orifices, such as nozzles. A fan or plurality of fans are connected to or in communication with the sides of the portal to facilitate the emission of the hydroxyl ions through the sides and ultimately through the orifices, such as nozzles, of the sides and/or top portion of the portal.

The portal can simply be two panels opposed to each other with nozzles pointed across from each other from the panels. In one aspect, a top portion is not required.

In another aspect, a top portion is part of the portal and helps to form an “arch” or a rectangular box through which an object can pass. In yet another aspect, the top portion can be in the form of a wedge such that the portal has an apex.

The side panels and/or the top portion of the portal can be manufactured from various materials including plastics, metals such as stainless steel, aluminum, etc. and is not limiting. In one aspect, the side panels and/or top portion are manufactured from sheet aluminum.

The dimensions of the portal can vary depending upon many factors including what type of facility or business the portal is to be located. Exemplary dimensions include side panel widths of from about 1 foot to about 6 feet, lengths of from about 1 foot to about 12 feet, e.g., about 1 foot, and depth of panel side from about 1 inch to about 2 feet, e.g., about 12 inches. The ultimate width of the side panel can be a result of the attachment of multiple side panels to each other.

Similarly, the top portion(s) can also have a width of from about 1 foot to about 6 feet, a length of about 1 foot to 12 feet and a depth of about 1 inch to about 2 feet, e.g., about 5 inches.

Orifices, for example nozzles, can be positioned in an array about the side panels and/or the top portion of the portal. The dimensions of the orifices or nozzles can vary but the orifice or nozzle opening is generally from about ½ inch to about 1 ½ inches, e.g., about 1 inch for the opening. The plurality of orifice(s) or nozzle(s) can be arranged in an array on the interior of the side panels and/or the top portion of the portal. It should be understood that the orifices can be positioned in linear fashion (e.g., in rows) or can be positioned randomly.

The distance between the plurality of orifices or nozzles can be varied to provide maximum delivery of hydroxyl ions through the orifices or nozzles to the interior section of the portal formed by the side panels and/or top portion. Typically, orifices or nozzles are positioned about 2 to 4 inches apart from each other linearly and from about 6 to about 12 inches from each other in adjacent rows. The number of orifices or nozzles per side panels can be varied depending on the dimensions of the side panels and/or the top portion. Typically, for a side panel of about 3 feet wide and 7 feet in length, there are about 64 orifices or nozzles positioned about the panel and there are about 40 orifices or nozzles for a 3 foot by 3 foot top portion.

The hydroxyl ions used with the apparatus described herein are generated by a hydroxyl ion generator. Hydroxyl ion generators are known in the art and include components such as UV (UVA) bulbs, a titanium oxide platform/support, titanium dioxide, and a water source to provide humidity. One or more fans are generally provided to direct the hydroxyl ion airstream into and through the portal system.

The UV source typically is a UVA bulb. One or more UV bulbs can be used in the hydroxyl ion generator to obtain maximum formation of hydroxyl ions.

Alternatively the light emitting source can be a light emitting diode (LED).

The hydroxyl generator creates hydroxyl radicals through a photocatalytic reaction utilizing UVA bulbs and titanium dioxide (TiO₂) coated onto or impregnated into perforated platforms, such as carbon fibers, ceramic panels, aluminum panels and the like. The process utilizes UVA (black light) in the 320 nm to 385 nm region, e.g., 365 nm, wavelength to excite (irradiate) nano sized titanium dioxide particles. Typical hydroxyl generators generate hydroxyls gas having a hydroxyl concentration of about 900 to about 1000 ppm.

The hydroxyl radical, •OH, is the neutral form of the hydroxide ion (OH—). Hydroxyl radicals are diatomic molecules that are highly reactive and very short-lived with an average half-life of less than two seconds. Hydroxyls work primarily by abstracting hydrogen atoms, thereby dismantling the molecular structure of volatile organic compounds (VOCs). The chain reaction caused by a cascade of organic oxidizing agents is stable enough to react with nearly all organic chemicals and many inorganic chemicals throughout the entire treatment space. The hydroxyl generator directs the contaminated air inside the unit where the ultraviolet rays purify the air and prevent pathogens, such as protozoa, fungi, molds, viruses, microorganisms, and other contaminants from multiplying again. As long as the system is in operation, chain reactions continue, ensuring a constant flow of hydroxyl ions.

The titanium dioxide is coated onto the platform/support. Alternatively, the titanium dioxide is impregnated into the platform.

In order for a hydroxyl generator to produce high levels of hydroxyl radicals, the humidity in the air needs to be about 60%. To avoid having to purchase and locate a separate humidification system, the embodiments described herein have the humidification system built in. In addition to having the humidification unit built into the apparatus, the system is focused on surface sanitation as opposed to air purification. Even though the unit is built to sanitize surfaces, it also has the added benefit of cleaning and purifying the air as well.

The humidification system can be, for example, an ultrasonic humidification system such as those known in the art. An ultrasonic humidifier is one that uses high-frequency sound vibrations to produce an extra fine water mist that is then expelled to add moisture to an area, in this case, in close proximity to the UV lights and the titanium oxide coated/impregnated support. The resulting moisture and hydroxyl ions are then passed through the side panel and/or top portion enclosures described herein. A fan or series of fans are generally positioned above or below the humidification/hydroxyl generator system to force the vapors through the side panel conduits/top portion conduit and ultimately through the orifices or nozzles.

The perforated platform/support for the titanium dioxide can be manufactured from a multitude of materials as noted above. The support can be a mesh or screen or any support with a porosity (hole size) sufficient to permit maximum airflow through the portal system/orifices or nozzles. Suitable porous supports include, for example, metallic or porcelain as described above with hole dimensions of from about 0.1 microns to about 5 centimeters. The hole does not need to be symmetrical and can be circular, oblong, trapezoidal, square, etc. Ideally, the perforated platform/support (coated with titanium dioxide) is configured so as to minimally disrupt the airflow of the system described herein.

Alternatively, the support can be flat or rod sheets/rods of metal.

As noted above, one or more fans are utilized to create an airflow through the apparatus and conduct the generated hydroxyl ions through the apparatus and through the multitude of orifices or nozzles. Suitable airflow volumes, cubic feet per minute (cfm), for the fan(s) are from about 600 to about 1000 cfm. In one aspect, there is a balance between the highest CFM possible to distribute the hydroxyl ions while keeping the airflow comfortable for people to pass through the apparatus and keeping the fans noise decibels at a comfortable level. Therefore, the apparatus is designed to provide maximum airflow while keeping the airflow comfortable, and the noise levels as low as possible.

In one aspect, the side panels and/or top portion are divided into two halves so that at least two hydroxyl generators are enclosed per side panel. The number of hydroxyl generator(s)/fan(s)/water source(s) should not be considered limiting as the number of components can vary dependent upon the overall size of the portal system. One, two, three, four, or more hydroxyl generators and the accompanying components can be housed in multiple side panels, for example, or in very large side panels with multiple “bays” that can include a separator portion every few feet, if necessary, to help keep the hydroxyl ion content maximized.

The water source(s) should be constructed as in line continuous water source(s). That is, water should be constantly supplied to a holding tank(s) in the hydroxyl generator/humidification system. The water should be passed through a filter system to remove any impurities that can damage the ultrasonic humidification system. Impurities can affect the generation of the hydroxyl ion and are therefore removed from the water source.

The water source/ultrasonic humidifier can include an automatic shut off valve to the water holding tank so that water does not overflow from the tank. Typical holding tank size is about 1 to about 5 gallons, e.g., 3.6 gallons and, as noted above, multiple tanks can be incorporated into the portal system depending on the width of the side panel(s). In one embodiment, the ultrasonic humidifier is positioned in the container that holds the water and is situated about 5 to about 30 mm below the water line in order to facilitate the humidification. In one embodiment, the humidification device can be attached a float that helps regulate the level of water in the container. The float can simply float within the water container or, alternatively, the float can be held in position by having one or more enclosed holes in the float which can slide up and down in relation to the water level within the water container. In another embodiment, the humidification device is located within the float.

Additionally, the air that is forced through the portal system is filtered as well to remove any particulates, etc. from the air. A high flow electrostatic air filter can be used to cleanse the air prior to entry into the portal system. The filter is typically incorporated into one or more of the side panels of the portal system.

In operation, generally, a fan is positioned above the hydroxyl generator (UV lamps, humidifier, and titanium oxide coated mesh) to pull through the hydroxyl ions into the portal system and orifices or nozzles. Alternatively, the fan can be positioned below the hydroxyl generator to push the hydroxyl ions through the portal system and orifices or nozzles.

In operation, the apparatus can provide a hydroxyl concentration of sufficient concentration to eliminate pathogens, including for example, protozoa, bacteria, fungi, molds, viruses, etc. (e.g., coronaviruses, such as COVID-19) upon exposure to the hydroxyl ions.

A smart tablet can be included with the apparatus which explains how the apparatus works and how to pass/walk an object through the apparatus system.

In another embodiment, an apparatus is provided that includes a directional air flow apparatus and a hydroxyl ion generator in communication with the directional air flow apparatus. The directional air flow apparatus can help support the components of the hydroxyl ion generator system within the conduit/directional air flow apparatus. A vapor of hydroxyl ions can be transmitted through the directional air flow apparatus to emit the vapor of hydroxyl ions into the surrounding area to kill or reduce the concentration of pathogens, such as protozoa, fungi, molds, viruses and/or bacteria in the air or surfaces in contact with the hydroxyl ion(s). It should be understood that the directional air flow apparatus is used as a housing to contain/support the hydroxyl ion generator system components and is not to be limiting in terms of impeding distribution of hydroxyl ions upon their generation due to the hydroxyl ion reactivity/activity being short lived.

The apparatus can be mounted from the ceiling or can be a standalone unit placed in an enclosed area. Multiple units can be used to treat larger areas.

Typically the surrounding area is an enclosed area such as a restaurant, hospital room, classroom, warehouse, office, etc. That is, the space generally does not have free flowing fresh air and is usually heated/cooled by a heating/ventilation/air conditioning system (HVAC).

The directional air flow apparatus can be ductwork, piping, tubing, and the like that can be configured to distribute the hydroxyl ions into the enclosed area. In one aspect, the directional air flow apparatus can be configured so that the hydroxyl ions are expelled from the hydroxyl generator into a first smaller volume/space that expands into a second larger volume/space of the apparatus, so that mixing of the hydroxyl ions and ambient air are sufficiently mixed as the hydroxyl ions dissipate throughout the enclosed space.

It should be understood that the apparatus expels and distributes hydroxyl ions into the air, killing or reducing the ill effects of protozoa, viruses, molds, fungi, bacteria, etc. and on surfaces in the areas where the hydroxyl ion is active/reactive.

Exposure of the pathogen(s) to hydroxyl ions via the apparatus’ and methods described herein can result in eradication of between about 40% to about 100%. For example, a reduction of viable pathogens after exposure to hydroxyl ions for 15 minutes can result in about 44% reduction. 30 minutes in about 80% reduction, 60 minutes in about 90% reduction and after 90 minutes greater than 90% reduction.

### EXAMPLES

### Objective

To determine the antibacterial efficacy of ionizing device against S.epidermidis after 15, 30, 60 and 90 minutes of exposure time at room temperature. Staphylococcusepidermidis (S.epidermidis) - ATCC 12228

### Test Method

### Bacterial Inoculum Preparation

For S.epidermidis, a pure culture was first plated onto tryptic soy agar supplemented with 5% sheep blood (TSAB) and incubated at 35° C. for 24 hours. A well-isolated colony was then harvested and plated onto fresh TSAB and incubated at 35° C. for 24 hours. Well-isolated colonies were then harvested, suspended in 10% Tryptic Soy Broth (TSB) and vortexed for 1 minute to ensure homogenization. This suspension was used to inoculate the test carriers (sterilized glass slides).

### Inoculation of the Test Carriers

Individual sterile glass carriers were inoculated with 50 µL of the microbial suspension and spread evenly across the carrier. This was repeated in triplicate for each time point and the controls. The inoculated carriers were then allowed to air dry (~45 minutes) inside a biological safety cabinet. Initial testing of the glass carriers showed starting concentrations of 6.60 x10⁶ cells/slide for S.epidermidis at time 0.

Simultaneously, the control slides were similarly prepared and inoculated. Inoculated test surfaces were placed one foot away from the ionizing device at right angle and subjected to 15, 30, 60 and 90 minutes of exposure time. Triplicate controls were performed to determine the untreated microbial populations on the test surfaces. After the exposure, the test carriers were placed into centrifuge tubes with sterile buffer water and vortexed to recover any remaining microbes. For S.epidermidis, the recovered samples were serially diluted, plated onto Petrifilm AC plates and incubated for 48 hours at 36±1° C. All test samples and untreated controls were performed in triplicate.

Recovery of Test Organisms: Following exposure, the entire inoculated test carriers and untreated controls were removed using pre-sterilized forceps and placed into 20 mL of phosphate-buffered saline (PBS). The samples were vortexed for 30-60 seconds to recover any remaining bacteria into suspension. The suspension was then serially diluted. For S.epidermidis, 1 mL of each dilution was plated onto AC Petrifilm plates and incubated at 35° C. for 24 hours. After incubation, the recovered colonies were counted. All tests were completed in triplicate.

Table 1. Quantitative counts for S.epidermidis exposed to ionizing devices and untreated control at different time points. The CFU results are based on the average of three Petrifilm counts.

Table 1

| Time Point | Sample | Exposure Time (min) | Bacterial Recovery CFU/Test Surface (average of 3 surfaces) | Log Reduction | % Reduction |
|---|---|---|---|---|---|
| 0 | Untreated control | 0 | 6,600,000 | | |
| 30 minutes | Untreated control | 0 | 2,000,000 | | |
| 90 minutes | Untreated control | 0 | 1,340,000 | | |
| 15 minutes | Treated Sample | 15 | 3,700,000 | 0.25 | 44 |
| 30 minutes | Treated Sample | 30 | 1,400,000 | 0.17 | 78 |
| 60 minutes | Treated Sample | 60 | 813,000 | 0.40 | 87 |
| 90 minutes | Treated Sample | 90 | 513,000 | 0.42 | 92 |

CFU: Colony forming Units, Detection limit = 10 CFU
% Reduction - Percent difference between untreated population and treated (exposed) population

The ionizing device was able to kill 87% of S.epidermidis bacteria after 60 minutes of treatment.

The following paragraphs enumerated consecutively from 1 through 34 provide for various aspects of the present invention. In one embodiment, in a first paragraph (1), the present invention provides an apparatus comprising:
a portal comprising a plurality of orifices arranged in an array and positioned on at least one of a first and/or a second opposing side panel of the portal; and
a hydroxyl ion generator in communication with the portal and the plurality of orifices, wherein a vapor of hydroxyl ions can be transmitted through the orifices to emit the vapor of hydroxyl ions into the area formed by the portal.

2. The apparatus of paragraph 1, wherein a first hydroxyl ion generator is in communication with the first side of the portal.
3. The apparatus of paragraph 1, wherein a second hydroxyl ion generator is in communication with the second side of the portal.
4. The apparatus of any of paragraphs 1 through 3, further comprising at least one fan in communication with either the first or second hydroxyl ion generator.
5. The apparatus of any of paragraphs 1 through 4, further comprising a top portion of the portal in communication with the first and second opposing side panels of the portal.
6. The apparatus of paragraph 5, wherein the top portion further comprises a plurality of orifices, wherein the vapor of hydroxyl ions can be transmitted through the top portion orifices.
7. The apparatus of any of paragraphs 1 through 6, further comprising at least one fan in communication with the first hydroxyl ion generator and at least one fan in communication with the second hydroxyl ion generator.
8. The apparatus of any of paragraphs 1 through 7, wherein the hydroxyl ion generator comprises a perforated mesh coated with titanium dioxide, a water source, a humidification system and a UV light generator that provides a spectrum of light from about 320 nm to about 385 nm.
9. The apparatus of any of paragraphs 1 through 8, wherein the water source is a continuous water source.
10. The apparatus of any of claims 1 through 9, wherein the water is stored in a container.
11. The apparatus of any of claim 1 through 10, further comprising an attached float to regulate the level of water in the container.
12. The apparatus of any of paragraphs 1 through 11, wherein the humidification system is an ultrasonic humidification system.
13. The apparatus of any of paragraphs 1 through 12, further comprising a water filter in communication with the water source and the humidification system.
14. The apparatus of any of paragraphs 1 through 13, further comprising a conveyor system in communication with the portal, wherein objects can be passed through the portal system on the conveyor system.
15. A method to disinfect a surface comprising the steps:
   passing an object through the apparatus of any of paragraphs 1 through 14, wherein the object is subjected to hydroxyl ions in an amount sufficient to reduce or eliminate one or more pathogens on the surface of the object.
16. The method of paragraph 15, wherein the humidification system provides at least 60% humidity for use with the UV light and titanium oxide to generate the hydroxyl ions.
17. The method of paragraph 15, wherein the object is an inanimate object or is a living being.
18. The method of paragraph 15, wherein the living being is an individual or a mammal.
19. The method of any of paragraphs 15 through 18, wherein the object is subjected to hydroxyl ions through the portal for a period of 1 second to 30 seconds.
20. The method of any of paragraphs 15 through 19, wherein the hydroxyl concentration is at a level that reduces or eliminates one or more pathogens upon exposure to the hydroxyl ions.
21, In another embodiment, an apparatus is provided comprising:
   a directional air flow apparatus; and
   a hydroxyl ion generator in communication with the directional air flow apparatus, wherein a vapor of hydroxyl ions can be transmitted through the air flow apparatus to emit the vapor of hydroxyl ions into the surrounding area.
22. The apparatus of paragraph 21, wherein the directional air flow apparatus is a conduit, ductwork, piping, or tubing.
23. The apparatus of either of paragraphs 21 or 22, wherein the directional air flow apparatus is configured to disperse the vapor or hydroxyl ions into an enclosed space.
24. The apparatus of any of paragraphs 21 through 23 further comprising at least one fan in communication with the hydroxyl ion generator.
25. The apparatus of any of paragraphs 21 through 24, wherein the hydroxyl ion generator comprises a perforated mesh coated with titanium dioxide, a water source, a humidification system and a UV light generator that provides a spectrum of light from about 320 nm to about 385 nm.
26. The apparatus of any of paragraphs 21 through 25, wherein the water source is a continuous water source.
27. The apparatus of any of paragraphs 21 through 26, wherein the water is stored in a container.
28. The apparatus of any of paragraph 21 through 27, further comprising an attached float to regulate the level of water in the container
29. The apparatus of any of paragraphs 21 through 28, wherein the humidification system is an ultrasonic humidification system.
30. The apparatus of any of paragraphs 21 through 29, further comprising a water filter in communication with the water source and the humidification system.
31. A method to disinfect a surface or air space comprising the steps:
   treating an enclosed area with the apparatus of any of paragraphs 21 through 30, wherein the enclosed area is subjected to hydroxyl ions in an amount sufficient to reduce or eliminate one or more pathogens throughout the air in the enclosed area and/or on the surface of objects within the enclosed area.
32. The method of paragraph 31, wherein the humidification system provides at least 60% humidity for use with the UV light and titanium oxide to generate the hydroxyl ions.
33. The method of any of paragraphs 21 through 32, wherein the hydroxyl concentration is at a level that reduces or eliminates one or more pathogens upon exposure to the hydroxyl ions.
34. The method of any of paragraphs 15 through 19 or 31 through 33, wherein the percentage reduction of one or more pathogens is between about 40% and 100%, e.g. 44% to about 92%, upon exposure to hydroxyl ion from between about 15 minutes to about 90 minutes.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Figure Identifiers

10 Portal apparatus system
20 Side panel(s) (left or right)
30 Optional divider/separator in side panel
35 Start of radius for top portion or angle for top portion
40 Fan
50 Mesh/support with titanium dioxide
70 Water container
80 Humidification device
90 In line water filter
100 In line water pressure reducer
110 In line water source
120 Air filter
130 Orifices in side panel 20
140 Top portion of portal apparatus system 10
150 Cut away view of side view of portal apparatus system 10
160 Humidification system including water container 70, in line water source 110, in line water pressure reducer 100, in line water filter 90 and humidification device 80.
170 Hydroxyl ions
180 Air stream
190 Interior portion of portal apparatus system 10

The following Figures provide a general embodiment of the walk through portal system described herein.

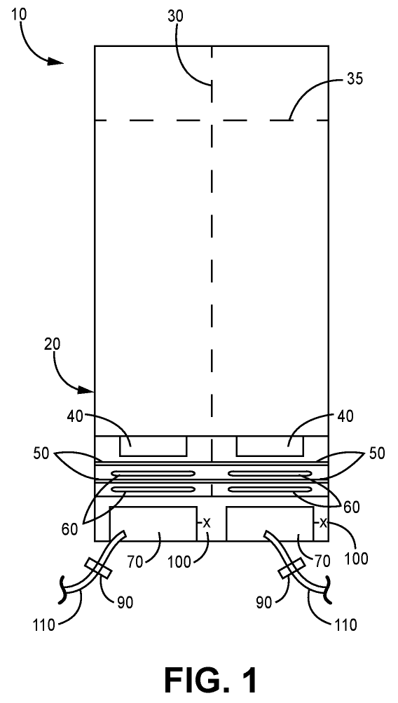

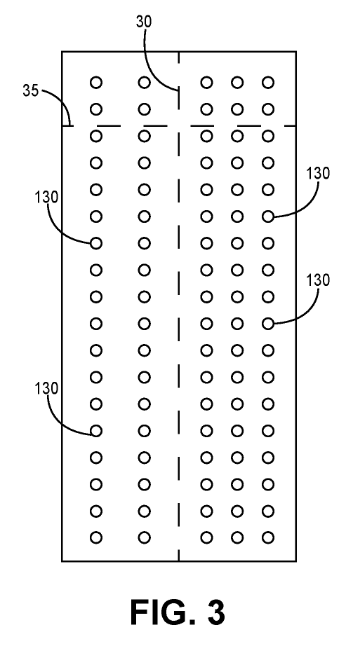

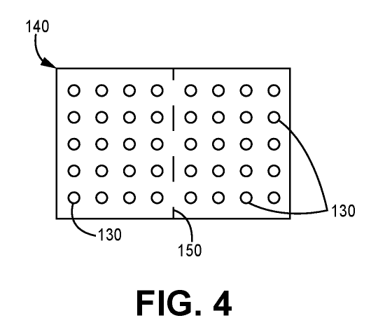

FIG. 1 is a cut away view of a portal apparatus 10 includes side panels 20 which can include an optional divider/separator 30 within the side panel 20. Panel 20 shows the bend for a radius or angle 35 for an optional top portion which can connect two side panels. Within side panel 20 there is contained a fan 40, a mesh support (with titanium dioxide) 50, a UV source (bulb) 60 and a water container 70. A humidification device 80, not shown, resides within water container 70 such that humidification device 80 is position between about 5 to about 3 mm below the water line. Optionally, humidification device 80 resides on or within a float (not shown) that helps regulate the water level in water container 70. Water container 70 is connected to an in line water source 110 which has an in line water filter 90 and also an in line water pressure reducer 100. In operation, the humidifier 80 generates water vapor which passes through/by mesh support 50 and UV source 60 to generate hydroxyl ions. The vapor with hydroxyl ions is directed through hollow side panel 20 to orifices 130 shown in FIG. 3 and FIG. 4.

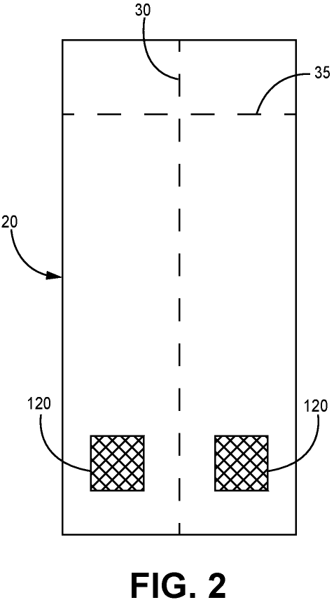

FIG. 2 provides an exterior view of hollow side panel 20 of portal apparatus system 10 with air filter 120.

FIG. 3 is an interior view of hollow side panel 20 of portal apparatus system 10 with orifices 130 (e.g., nozzles) spread in an array.

FIG. 4 depicts an optional top portion 140 of portal apparatus system 10 with orifices 130 (e.g., nozzles) spread in an array and an optional point of inflection 150 for a maximum radius or point to top portion 140.

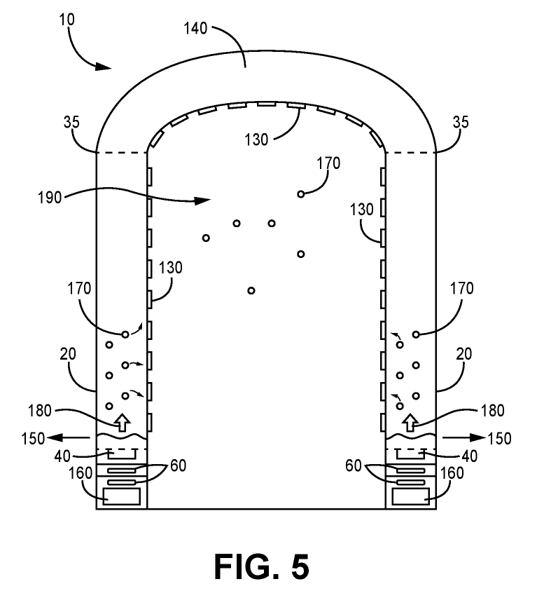

FIG. 5 depicts a side view of portal apparatus system 10 (not to scale) showing side panels 20, the start of radius 35 to optional top portion 140 and orifices 130 (shown as nozzles and not drawn to scale). Cut away section 150 depicts fan 40, mesh support 50, UV source 60 and humidification system 160. Fan 40 creates an air stream 180 to cause hydroxyl ions 170 to flow into the interior 190 of portal apparatus system 10 through orifices 130.

### Figure Identifiers

300 Directional air flow apparatus
310 Cut away for hydroxyl ion generator components
320 Optional flange to facilitate dispersion of hydroxyl ions
330 Air flow
340 Hydroxyl ion(s)
350 HEPA filter
360 Water container
370 Water pressure reducer
380 Water line
390 UVA bulb
400 Perforated support with titanium oxide (TiO₂) coating
410 Fan

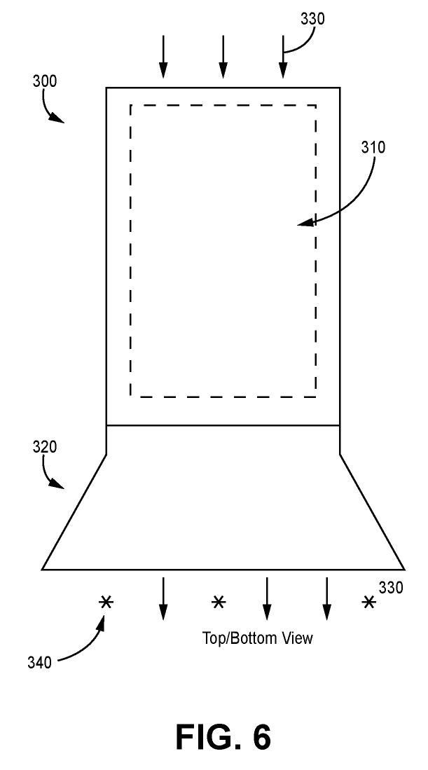

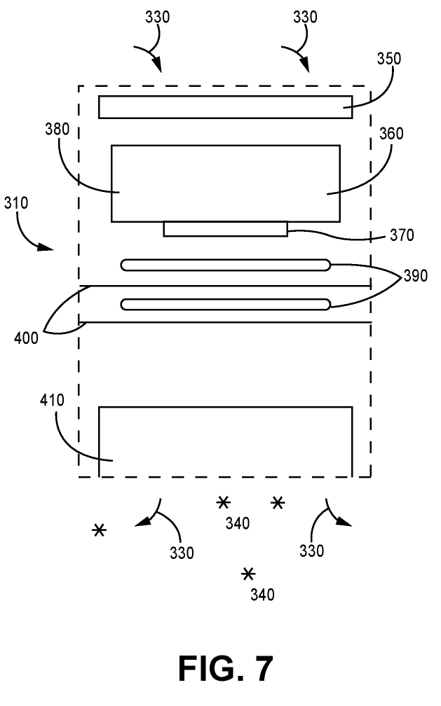

FIG. 6 is a top or bottom view of a stand-alone apparatus suitable for use in an enclosed area. The apparatus can be mounted to a ceiling or can be positioned in a convenient location within the enclosed space. FIG. 6 shows directional air flow apparatus 300 with an optional flange 320 in communication with the directional air flow apparatus 300. 310 is a cut away view of hydroxyl ion generator components for use with the apparatus described in FIG. 7.

FIG. 7 is a top or bottom view of cutaway view 310 of FIG. 6. The hydroxyl ion generator components include with directional air flow apparatus 300, a HEPA (high efficiency particulate air) filter 350. Air flow 330 enters directional air flow apparatus 300 drawn by fan 410 to emit hydroxyl ions 340 from the unit. Air flow 330 passes over water container 360, equipped with water pressure reducer 370 and an ultrasonic device (not shown), optionally with a float (not shown), through UVA bulbs 390 and perforated support with a TiO₂ coating 400 and expelled from directional flow apparatus 300 through optional flange 320 to emit activated hydroxyl ions 340 into an enclosed area.

In one aspect, fan 420 can be positioned in front of HEPA filter 350 to push air through directional air flow apparatus instead of drawing air through the apparatus.

In another aspect, the apparatus can be integrated with an HVAC system to purify the incoming air within the enclosed space.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

1-34. (canceled)

35. An apparatus comprising:
a portal comprising orifices positioned on at least one of a first and/or a second opposing side panel of the portal; and
a hydroxyl ion generator in communication with the portal and the plurality of orifices, wherein a hydroxyl ions are transmitted through the orifices to emit hydroxyl ions into the area formed by the portal.

36. The apparatus of claim 35, wherein a first hydroxyl ion generator is in communication with the first side of the portal.

37. The apparatus of claim 35, wherein a second hydroxyl ion generator is in communication with the second side of the portal.

38. The apparatus of claim 35, further comprising at least one fan in communication with either the first or second hydroxyl ion generator.

39. The apparatus of claim 35, further comprising a top portion of the portal in communication with the first and second opposing side panels of the portal.

40. The apparatus of claim 39, wherein the top portion further comprises a plurality of orifices, wherein the hydroxyl ions can be transmitted through the top portion orifices.

41. The apparatus of claim 35, further comprising at least one fan in communication with the first hydroxyl ion generator and at least one fan in communication with the second hydroxyl ion generator.

42. The apparatus of claim 35, wherein the hydroxyl ion generator comprises a perforated mesh coated with titanium dioxide, a water source, a humidification system and a UV light generator that provides a spectrum of light from about 320 nm to about 385 nm.

43. The apparatus of claim 42, wherein the water source is a continuous water source.

44. The apparatus of claim 42, wherein the water is stored in a container.

45. The apparatus of claim 44, further comprising an attached float to regulate the level of water in the container.

46. The apparatus of claim 42, wherein the humidification system is an ultrasonic humidification system.

47. The apparatus of claim 35, further comprising a conveyor system in communication with the portal, wherein objects can be passed through the portal system on the conveyor system.

48. A method to disinfect a surface comprising the steps:
passing an object or a person through the apparatus of claim 35, wherein the object or person is subjected to hydroxyl ions in an amount sufficient to reduce or eliminate one or more pathogens on the surface of the object or person.

49. An apparatus comprising:
a directional air flow apparatus; and
a hydroxyl ion generator in communication with the directional air flow apparatus, wherein hydroxyl ions are transmitted through the directional air flow apparatus to emit the hydroxyl ions into the surrounding area.

50. The apparatus of claim 49, wherein the directional air flow apparatus is a conduit, ductwork, piping, or tubing.

51. The apparatus of claim 49, wherein the directional air flow apparatus is configured to disperse the hydroxyl ions into an enclosed space.

52. The apparatus of claim 49, further comprising at least one fan in communication with the hydroxyl ion generator.

53. The apparatus of claim 49, wherein the hydroxyl ion generator comprises a perforated mesh coated with titanium dioxide, a water source, a humidification system and a UV light generator that provides a spectrum of light from about 320 nm to about 385 nm.

54. The apparatus of claim 53, wherein the water source is a continuous water source.

55. The apparatus of claim 53, wherein the water is stored in a container.

56. The apparatus of claim 55, further comprising an attached float to regulate the level of water in the container.

57. The apparatus of claim 53, wherein the humidification system is an ultrasonic humidification system.

58. A method to disinfect a surface or air space comprising the steps:
treating an enclosed area with the apparatus of claim 15, wherein the enclosed area is subjected to hydroxyl ions in an amount sufficient to reduce or eliminate one or more pathogens throughout the air in the enclosed area and/or on the surface of objects within the enclosed area.
